(19) United States
(12) Patent Application Publication
(10) Pub. No.: US 2009/0030413 A1
(43) Pub. Date: Jan. 29, 2009

(54) Surgical instrument and method for treating scar encapsulation

(76) Inventor: Zachary E. Gerut, Hewlett, NY (US)

Correspondence Address: BERNARD MALINA, ESQ., MALINA & ASSOCIATES, PLLC, 60 East 42nd Street, Suite 501, New York, NY, 10165, US

(21) Appl. No.: 11/881,715

(22) Filed: Jul. 27, 2007

(51) Int. Cl. A61B 18/14

(52) U.S. Cl. 606/49

(57) ABSTRACT

A surgical instrument and method of use is described for the treatment of scar encapsulation, particularly related to malleable implants, such as silicone or saline breast implants. The instrument includes an elongate housing with distal and proximal ends. At least two interior conduits traverse the housing. One of the at least two interior conduits splits into a plurality of smaller passages at or near the distal end permitting flow of a fluid or application of suction. A central one of the at least two interior conduits is an energy conduit fitted with an electrode.

### BACKGROUND OF THE INVENTION

1. Field of the Invention

The invention concerns a surgical instrument and method employing the instrument for treatment of scar encapsulation.

2. The Related Art

Capsular contracture around breast prostheses is the principal cause for dissatisfaction in breast augmentation. All breast prosthesis are soft, pliable and adjust to a pleasing shape when first installed; but the body's response to the presence of the implant may later cause contracture. The contributing factors of infection, inflammation, hematoma formation and unknown factors all lead to increased scar formation. Attempts to alter this basic response have been diverse and evolving over many years.

U.S. Pat. No. 4,955,909 (Ersek et al.) discloses overcoming the problem of capsular contracture arising from soft silicone implants by texturing the silicone surface of the prosthesis. This is found to lessen scar tissue formation. Unfortunately, the problem is not totally eliminated.

Another approach involves a subsequent surgical treatment to remove scar tissue. U.S. Pat. No. 5,425,355 (Kulick) reports a treatment with a surgical instrument that delivers an energy discharge to the affected area. Still further improvements are necessary to advance the art.

Accordingly, it is an object of the present invention to provide a surgical instrument which allows treatment of scar encapsulation, particularly tissue damage as a result of silicone or saline breast implants.

Further, an object of the present invention is to provide a method to treat scar encapsulation that eliminates or at least ameliorates implant deformity, hardness to the touch, and physical symptoms of pain and tenderness.

Still further, an object of the present invention is to treat scar encapsulation with a surgical instrument characterized by requiring only local anesthesia, minimal equipment for reduced cost, a relatively short procedure, and the delivery of therapeutic drugs for prevention of future encapsulation.

### SUMMARY OF THE INVENTION

A surgical instrument is provided which includes:
an elongate housing having distal and proximal ends, the distal end being blunt;
at least two interior conduits traversing the housing;
one of the at least two interior conduits splitting into a plurality of smaller passages at or near the distal end permitting flow of a fluid or application of suction; and
a central one of the at least two interior conduits being an energy delivery conduit fitted with an electrode.

Further, a method for treating scar encapsulation is provided which includes:
(i) providing a surgical instrument which includes:
   an elongate probe housing having distal and proximal ends, the distal end being blunt;
   at least two interior conduits traversing the housing;
   one of the at least two interior conduits splitting into a plurality of smaller passages at or near the distal end permitting flow of a fluid or application of suction; and
   a central one of the at least two interior conduits being an energy delivery conduit fitted with an electrode;
(ii) applying an end of the electrode against an area on a person's skin requiring remediation of a scar tissue and then delivering energy to that area;
(iii) delivering the fluid through the plurality of smaller passages to the area requiring remediation.

### BRIEF DESCRIPTION OF THE DRAWING

Further advantages and features of the present invention will become apparent from consideration of the following drawing in which:

FIG. 1 is a schematic cross-sectional view of a surgical instrument according to the present invention;

FIG. 2 is a cross-sectional view along line II-II of FIG. 1;

FIG. 3 is a cross-sectional view along line III-III at distal end of FIG. 1; and

FIG. 4 is a second embodiment of the plurality of smaller passages shown in FIG. 3.

### DETAILED DESCRIPTION OF THE INVENTION

Now there has been found an improved surgical instrument which allows treatment of scar encapsulation surrounding malleable implants such as silicone or saline breast implants. The instrument is inserted into a very small skin incision. Design of the instrument allows easy passage through subcutaneous tissue by use of a distal end energy discharge and a fluid discharge. Scar encapsulation surrounding malleable implants can thereby be effectively treated using only local anesthesia, minimal equipment, in less time and with greater efficacy. Therapeutic substances can also be applied through the instrument for prevention of future scar encapsulation.

Fluids of the present invention primarily are anesthetics. Additives may be incorporated into these fluids. Of particular interest are additives which are therapeutic drugs.

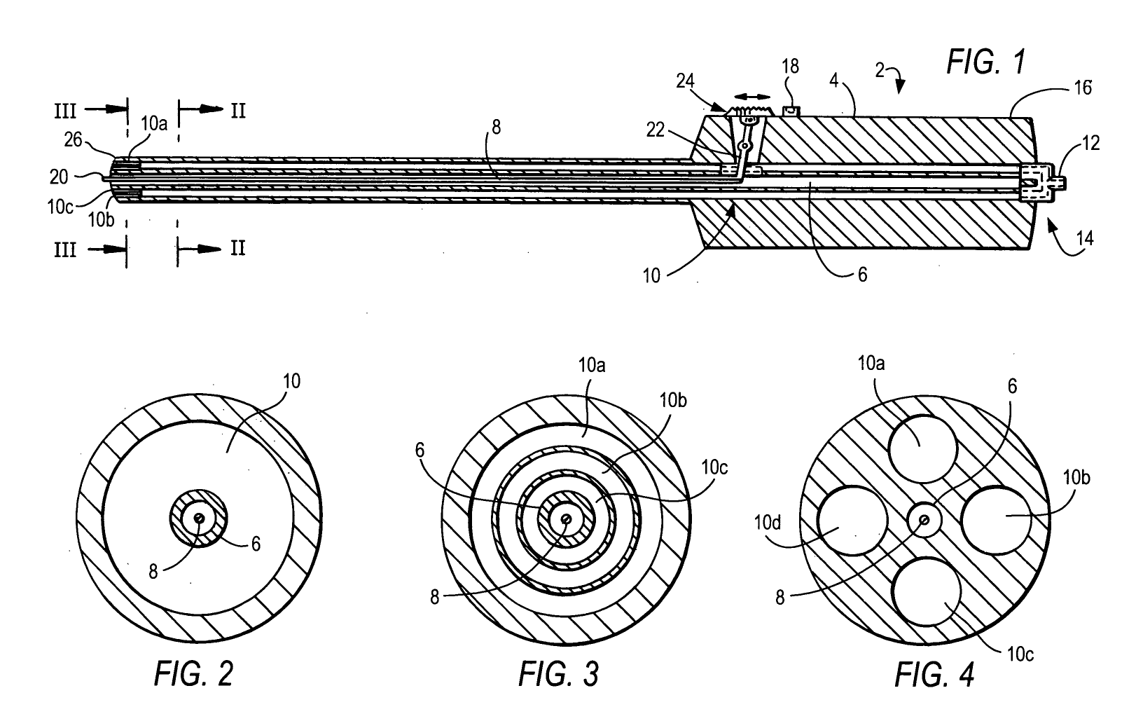

FIG. 1 illustrates the surgical instrument 2 in a cross-sectional schematic view. A housing 4 is formed around a central conduit 6 which is traversed by an electrode 8. A fluid conduit 10 concentrically surrounds the central conduit. Fluid or suction can be fed into coupling 12 at the proximal end 14 of the instrument.

Electrical energy is fed into a handle 16 of the instrument through connector 18. This electrical energy is converted through resistance in the electrode into heat energy expressed at electrode end 20. Movement along the energy delivery central conduit 6 by the electrode 8 can manually be manipulated by an advancement lever 22 through manual control of a flange or pressure button 24 attached to the handle 16. The electrode 8 can be advanced relative to a blunt tip 26 from a recessed position to a slightly protuberant position through activation of the flange or pressure button. In its most advanced position, the electrode at end 20 can be moved beyond blunt end 26 of the instrument. This advanced position allows energy (heat) dissection to be applied to less fragile soft tissues such as scar tissue. FIG. 2 illustrates in cross-section the concentric arrangement of electrode bearing conduit 6 and fluid conduit 10. Towards the distal end of the surgical instrument near tip 26, the fluid conduit 10 splits from a single channel into a plurality of smaller passages. In the illustrated embodiment shown in FIG. 3, these smaller passages are identified by numerals 10a, 10b, and 10c. A second more preferred embodiment is illustrated in FIG. 4. Therein, the plurality of small passages 10a, 10b, 10c, and 10d for fluid are merely apertures traversing a wall forming the distal blunt tipped end. The number of apertures is not intended to be limiting but could range from about 3 to about 200, preferably from about 5 to about 100, optimally from about 10 to about 20 apertures. Suction as well as fluid can be applied through the fluid conduit 10.

It will be understood that this specification is exemplary and the invention herein is to be liberally construed within the scope of the attached claims.

What is claimed is:

1. A surgical instrument comprising:
an elongate housing having distal and proximal ends, the distal end being blunt;
at least two interior conduits traversing the housing;
one of the at least two interior conduits splitting into a plurality of smaller passages at or near the distal end permitting flow of a fluid or application of suction; and
a central one of the at least two interior conduits being an energy delivery conduit fitted with an electrode.

2. The instrument according to claim 1 physically suitable for treatment of scar encapsulation of malleable implants.

3. The instrument according to claim 2 wherein the malleable implants are silicone or saline breast implants.

4. The instrument according to claim 1 wherein the fluid comprises an anesthetic.

5. The instrument according to claim 1 wherein the fluid comprises a therapeutic substance for treatment or prevention of future scar encapsulation.

6. The instrument according to claim 1 wherein the electrode is an electrocautery device.

7. The instrument according to claim 1 wherein the electrode is connected at the proximal end to an energy generation machine.

8. The instrument according to claim 7 wherein the energy generation machine heats the electrode.

9. The instrument according to claim 1 wherein the electrode is moveable from a recessed position interior of the housing at the distal end to a protuberant position where a leading tip of the electrode is outside the housing at the distal end.

10. The instrument according to claim 9 further comprising a mechanism at the proximal end for advancing and retracting the electrode along the energy delivery conduit.

11. The instrument according to claim 1 wherein the smaller passages are apertures traversing a wall forming the distal blunt end.

12. A method for treatment of scar encapsulation comprising:
(i) providing a surgical instrument which comprises:
an elongate probe housing having distal and proximal ends, the distal end being blunt;
at least two interior conduits traversing the housing;
one of the at least two interior conduits splitting into a plurality of smaller passages at or near the distal ends permitting flow of a fluid or application of suction;
a central one of the at least two interior conduits being an energy delivery conduit fitted with an electrode;
(ii) applying an end of the electrode against an area on a person's skin requiring remediation of a scar tissue and then delivering energy to that area; and
(iii) delivering the fluid through the plurality of smaller passages to the area requiring remediation.
